(19) United States
(12) Patent Application Publication
(10) Pub. No.: US 2026/0026820 A1
(43) Pub. Date: Jan. 29, 2026

(54) Embolic Coil Implant System

(71) Applicant: Piezaolabs Medical Inc., Lake Forest, CA (US)

(72) Inventor: Ayman Gerges Saweres Mina, Dubai (AE)

(73) Assignee: Piezolabs Medical Inc., Lake Forest, CA (US)

(21) Appl. No.: 18/787,728

(22) Filed: Jul. 29, 2024

(51) Int. Cl. A61B 17/12

(52) U.S. Cl. CPC A61B 17/1214; A61B 17/12113; A61B 2017/12054

(57) ABSTRACT

A delivery system for an embolic coil has a hypotube having a proximal end that is connected to the handle housing, with the hypotube having a lumen extending from the proximal end to a distal end of the hypotube. The lumen has a first diameter and a widened section that has a second diameter that is greater than the first diameter. A first wire has a first enlarged end provided at the proximal end of the first wire and an embolic coil provided at the distal end of the first wire. A second wire has a second enlarged end provided at the distal end of the second wire and with the proximal end of the second wire connected to the pivotable element. The wires and their enlarged ends extend inside the lumen of the hypotube. Pulling the second wire in a proximal direction causes the first and second enlarged ends to disengage when the first and second enlarged ends are both in the widened section.

### BACKGROUND OF THE INVENTION

### 1. Field of the Invention

The present invention relates to the fields of systems and methods for implanting an intravascular implant device, and more specifically to systems and methods for implanting embolic coils.

### 2. Description of the Prior Art

Hemorrhagic intracranial stroke due to aneurysm rupture is the leading cause of disability and/or death worldwide. Endovascular procedures using intracranial coils for treating a cerebral aneurysm now is the gold standard and fast-growing procedure, among other techniques, due to the ease of technique and reasonable cost.

A cerebral aneurysm is a common cerebrovascular disorder caused by a weakness in the wall of a cerebral artery. The disorder may result from congenital defects or from preexisting conditions, such as hypertensive vascular disease and atherosclerosis, or from head trauma.

Approximately 4% to 6% of the U.S. population is believed to harbor an intracranial aneurysm. It has been reported that there are between 45,000 and 50,000 annual intracranial aneurysm ruptures in North America, with a resultant combined morbidity and mortality rate of about 50%.

Rupture of a cerebral aneurysm is dangerous and typically results in bleeding in the brain or in the area surrounding the brain, leading to an intracranial hematoma.

Treating cerebral aneurysms requires not only high-quality more thrombogenic coils but also a reliable delivery system with almost zero risk of either pre-mature detachment or no detachment.

A typical occlusion coil is a wire coil having an elongate primary shape with windings coiled around a longitudinal axis. In the aneurysm coil embolization procedure, a catheter is introduced into the femoral artery and navigated through the vascular system under fluoroscopic visualization. The coil in the primary shape is positioned within the catheter. The distal end of the catheter is positioned at the site of an aneurysm within the brain. The coil is passed from the catheter into the aneurysm. Once released from the catheter, the coil assumes a secondary shape selected to optimize filling of the aneurysm cavity. Multiple coils may be introduced into a single aneurysm cavity for optimal filling of the cavity. The deployed coils serve to block blood flow into the aneurysm and reinforce the aneurysm against rupture.

One form of delivery system used to deliver an embolic coil through a catheter to an implant site includes a wire and a coil attached to the wire. The coil (with the attached wire) is advanced through a catheter as discussed above. To release the coil into an aneurysm, current is passed through the wire, causing electrolytic detachment of the coil from the wire. Other detachment systems use mechanical detachment to separate the coil from the wire.

These systems and detachment mechanisms suffer from a number of drawbacks.

For example, electrolytic detachment uses electrical current to generate thermal energy to melt the polymer that used to fix the coil implant to the delivery system. This melting action takes time, and this time delay could be critical if the aneurysm ruptured and bleeding starts.

Mechanical detachment systems have a number of different drawbacks. For example, some mechanical detachment systems require separate detachment handles which translates to greater cost and complexity. Technical failures have also been observed due to a missed connection of the delivery system to the detachment handle. In addition, the mechanical detachment designs that use wire finger snip technique could accidentally retrieve the last loop of the coil due to an applied longitudinal force that leads to collapse of last loop at the parent artery.

The present invention provides an alternative to prior art detachment systems that overcomes the drawbacks associated with these systems.

### SUMMARY OF THE DISCLOSURE

It is an object of the present invention to provide a delivery system for embolic coils which quickly and effectively detaches the coil from the wire while minimizing risks to the patient.

To meet the objectives of the present invention, there is provided a delivery system for an embolic coil, having a handle housing having a pivotable element that has a front end, and a hypotube having a proximal end that is connected to the handle housing, with the hypotube having a lumen extending from the proximal end to a distal end of the hypotube, where the lumen has a first diameter and a widened section that has a second diameter that is greater than the first diameter. A first wire has a first enlarged end provided at the proximal end of the first wire and an embolic coil provided at the distal end of the first wire. A second wire has a second enlarged end provided at the distal end of the second wire and with the proximal end of the second wire connected to the pivotable element. The first wire and its first enlarged end extend inside the lumen of the hypotube, and the second wire and its enlarged end extend inside the lumen of the hypotube, with the first and second enlarged ends engaging each other. The pivotable element is manipulated to pull the second wire in a proximal direction to cause the first and second enlarged ends to disengage when the first and second enlarged ends are both in the widened section.

In one embodiment, each of the first and second enlarged ends is an olive-shaped end. Each olive-shaped end has a longitudinal dimension and a width, and the longitudinal dimension is greater than the width, and wherein each olive-shaped end has a completely smooth surface.

In another embodiment, the lumen further includes a distal narrowed section and a proximal narrowed section, with the widened section positioned between the distal narrowed section and the proximal narrowed section, and wherein the first and second enlarged ends engage each other at the distal narrowed section during delivery of the embolic coil.

### BRIEF DESCRIPTION OF THE DRAWINGS

FIG. 1 is a schematic view of a delivery system for embolic coils according to one embodiment of the present invention.

FIG. 2 is an enlarged cross-sectional view of the hypotube of the delivery system of FIG. 1.

FIG. 3 is a proximal view of the hypotube of FIG. 2.

FIG. 4A is a perspective three-dimensional view of an olive-shaped enlarged end that is connected to a filament.

FIG. 4B is a side view of the enlarged end of FIG. 4A.

FIG. 5 is an enlarged cross-sectional view of the hypotube section of the delivery system of FIG. 1 showing the initial impact of a retrieving wire on the two enlarged ends positioned inside the retaining seat.

FIG. 6 is an enlarged cross-sectional view of the hypotube section of the delivery system of FIG. 1 showing the enlarged end for the filament that holds the coil being released from the retaining seat.

FIG. 7 is a schematic view of the delivery system showing how pressing the lever releases the coil.

FIG. 8A illustrates the forces involved when two spherical enlarged ends are pulled in opposite directions.

FIG. 8B illustrates the forces involved when two olive-shaped enlarged ends are pulled in opposite directions.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

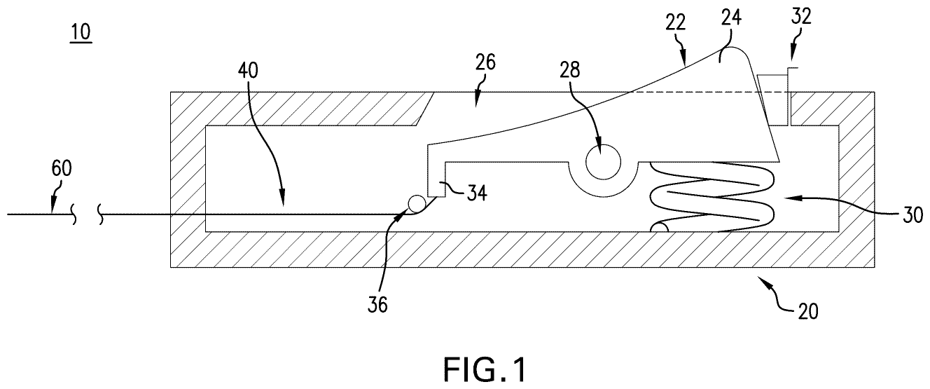

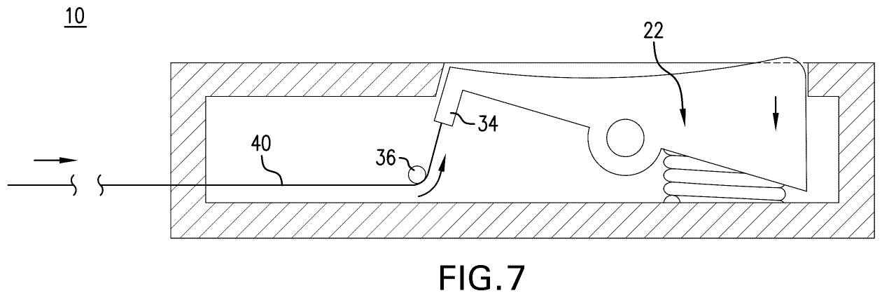

The delivery system 10 is shown in FIGS. 1 and 7, and has a handle housing 20 that houses a pivoting lever 22. The lever 22 has an upper grip portion 24 that extends out of the housing 20 through an opening 26. The lever 22 is pivotably secured inside the housing 20 via a connecting pin 28 that acts as a fulcrum. A spring 30 is secured to the rear bottom surface of the lever 22 and the base of the housing 20 to normally bias the rear of the lever 22 out of the opening 26. An optional security lock 32 can be provided at the rear of the opening 26 to lock the lever 22 in a fixed position.

The lever 22 has a hooked front end 34 that connects to the proximal end of a detachment filament or wire 40. The distal end of the wire 40 is connected to a first enlarged end 42. A roller 36 is provided inside the housing 20 adjacent the location of the front end 34 to smoothen the movement of the wire 40 as it is pulled or released by the lever 22.

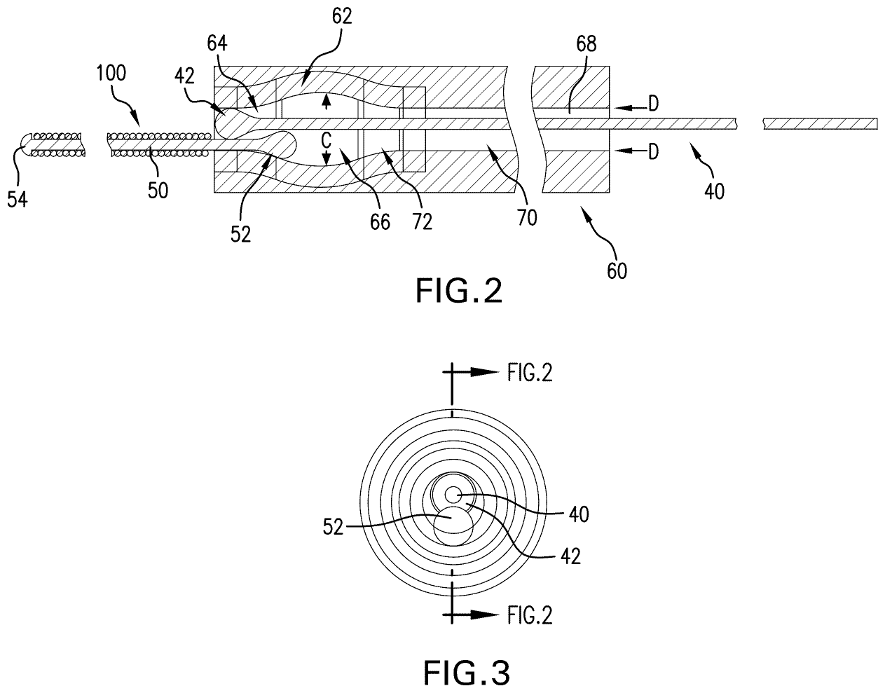

Referring now to FIGS. 2-6, the embolic coil 100 that is to be delivered to the aneurysm is wrapped around the distal end of a second filament or wire 50 that could be embodied as a stretch-resistant element. The proximal end of the wire 50 is connected to a second enlarged end 52 and the distal end of the wire 50 can be configured as a rounded atraumatic tip 54 of the coil 100. The two enlarged ends 42 and 52 are adapted to be engaged inside a distal narrowed section 64 of the retaining seat 62 that is defined inside a hyptotube 60. As explained in greater detail below, the two enlarged ends 42 and 52 function to prevent each from passing or crossing the other inside the narrowed section 64 of the retaining seat 62, while being allowed to pass or cross each other at a widened section 66 of the retaining seat 62.

The wire 50 can be made of soft elastic polypropylene materials, but the wire 40 should be made of non-stretchable materials such as nickel-titanium, stainless steel or any other non-stretchable material, to ensure the accurate transition of force applied the pushing upper grip portion 24 downwardly to retrieve or pull the wire 40 and its enlarged element 42 proximally for detachment.

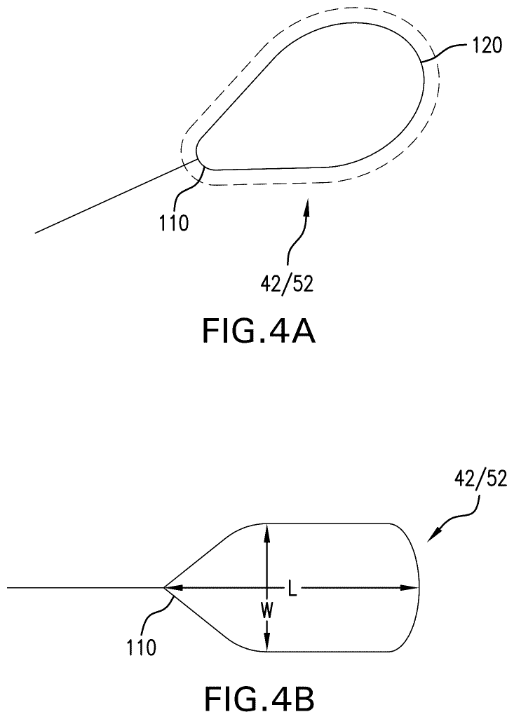

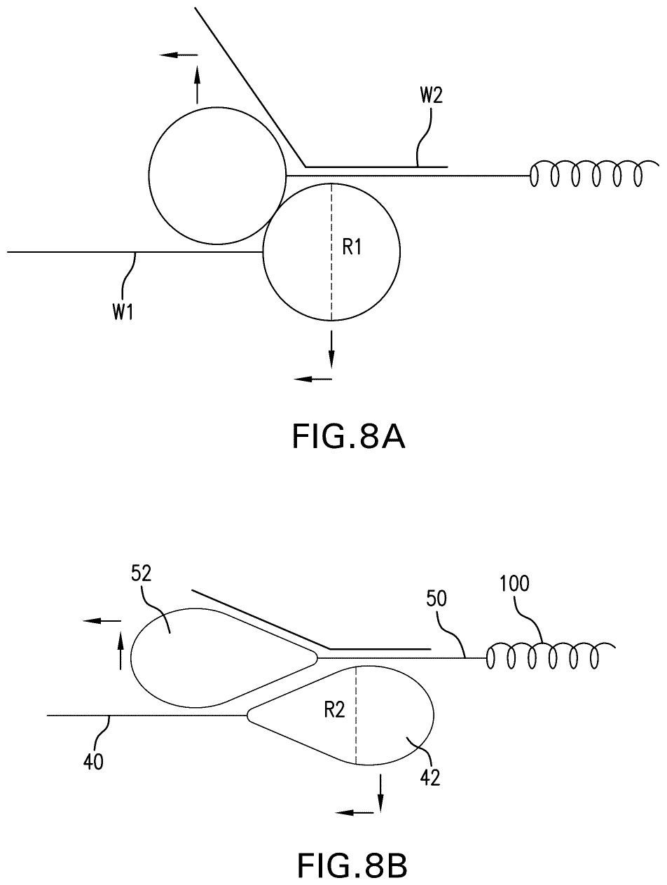

Each enlarged end 42 and 52 can have the same or different size, and are provided in an olive shape. “Olive” shape is defined herein as having an outer surface that is completely smooth with an elongation similar to that of an oval shape but not exactly like a true oval shape. More specifically, the olive shape is longer in a longitudinal direction L (see FIG. 4B) than in a width direction W, and having one end 110 that is more pointed and the other end 120 that is more rounded (see FIG. 4A). By “completely smooth”, it is meant that there are no edges or sharp points such that the outer surface essentially has a curvature throughout. In contrast to a sphere or oval, the outer surface of an olive is not uniform. In the present invention, the more rounded end 120 is free and the pointed end 110 is connected to the wire 40/50. As explained below in FIGS. 8A and 8B, this configuration allows for the applied pulling force to be more efficient in FIG. 8B compared to FIG. 8A where the wire 40 is closer to the wire 50 (along parallel lines) compared to the wires W1 and W2 in FIG. 8B.

The hypotube 60 preferably extends from the housing 20 to the location where the coil 100 detaches, for a total length of about 160 cm-250 cm, and has a lumen 70 extending therethrough. The lumen 70 has a proximal lumen section 68 with a consistent inner diameter throughout that extends from the housing 20 to a proximal narrowed section 72. The widened section 66 is between the distal narrowed section 64 and the proximal narrowed section 72. The retaining seat 62 includes the three sections 64, 66 and 72. The widened section 66 can have an annular curved inner wall that has an enlarged diameter compared to the lumens in all the other sections 64, 72 and 68. The lumen inside the widened section 66 is slightly wider than the sum of the widths of the two enlarged ends 42 and 52 (i.e., largest width of the lumen in the widened section 66>2W). In the narrowed sections 64 and 72, the diameter of the lumen 70 is greatest adjacent the widened section 66 and gradually decreases to the same diameter as the lumen at the proximal lumen section 68. As a result, the lumen 70 has its smallest diameter D along the proximal lumen section 68 and the proximal end of the proximal narrowed section 72, and the diameter of the lumen 70 gradually increases to its largest diameter C at the center of the widened section 66 before gradually decreasing to its smallest diameter D at the distal end of the distal narrowed section 64. This configuration for the lumen 70 allows the two enlarged ends 42 and 52 to be secured in an interlocking manner inside the widened section 66 as the combined widths (2W) of the two enlarged ends 42 and 52 is greater than the diameter of the lumen 70 at the narrowed sections 64 and 72 so that the enlarged ends 42 and 52 cannot escape the lumen 70 at either narrowed sections 64 and 72.

When the coil 100 is delivered to the target location inside the patient's vasculature, the enlarged ends 42 and 52 are interlocked inside the distal narrowed section 64. This is best illustrated in FIG. 2.

When the enlarged ends 42 and 52 are interlocked inside the retaining seat 62, there are two forces that act on this interlocking structure. The first force is the tension force of the coil implant 100 on the enlarged end 52 and the second force is the force of the wire 40 on the enlarged end 42 that is applied by the lever 22.

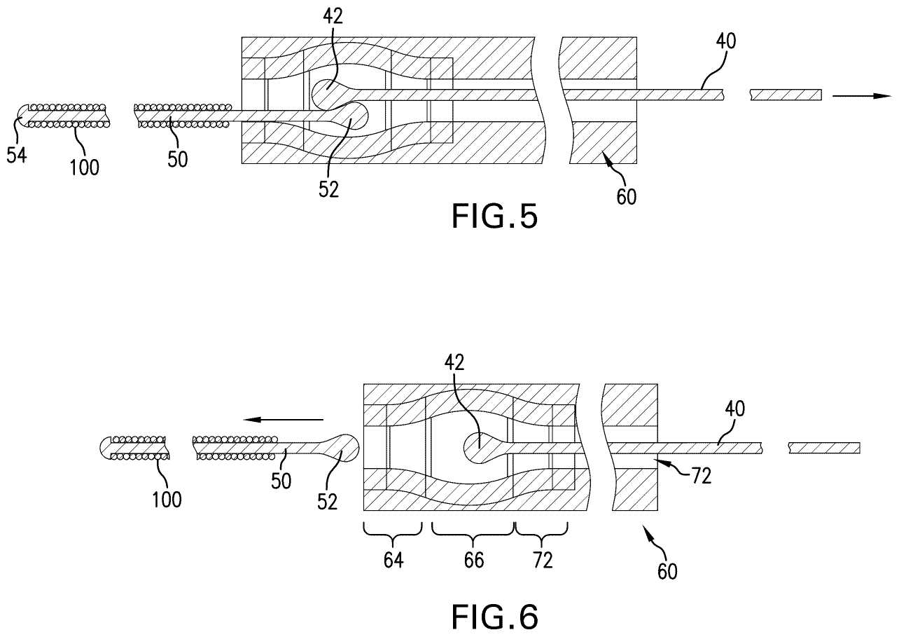

Once the operator is ready to detach the coil 100 after alignment with microcatheter markers, the operator will press or push the lever 22 down. See FIG. 7. This raises the front end 34, thereby pulling the wire 40 around the roller 36, which in turn pulls the enlarged end 42 in the proximal direction towards the widened lumen inside the widened section 66. The interlocking relationship between the enlarged ends 42 and 52 causes the enlarged end 42 to pull the enlarged end 52 towards the widened lumen inside the widened section 66. See FIG. 5. At the largest diameter C, there is enough space for the enlarged ends 42 and 52 to disengage, and when the hypotube 60 is retracted, the enlarged end 52 can pass through the lumen 70 at the distal narrowed section 64, and exit the hypotube 60. See FIG. 6. This releases the coil 100 that is carried on the wire 50.

FIGS. 8A and 8B illustrate how the olive-shaped enlarged ends 42 and 52 are more effective than conventional spherical or hemispherical enlarged ends. FIG. 8A illustrates the interlock between two spherical enlarged ends while FIG. 8B illustrates the interlock between two olive-shaped enlarged ends. There are two distinct advantages provided by the two olive-shaped enlarged ends.

First, as shown in FIGS. 8A and 8B, the sphere has a diameter R1 that is bigger than the width R2 for an olive shape, so that 2R1 is much greater than 2R2. This means that the largest width/diameter C at the center of the widened section 66 must be much larger if a spherical enlarged end is used instead of an olive-shaped enlarged end. Also, it is important to provide both enlarged ends 42, 52 inside an enclosed section of the hypotube 60 during delivery of the coil 100 to the site of the aneurysm. Therefore, the smaller width/diameter C would facilitate the provision of a hypotube 60 that can have a smaller diameter.

Second, the respective movement of the respective enlarged ends in opposing directions is smoother for the olive-shaped ends. In FIG. 8A, when the wire W1 is pulled in an opposing direction from the wire W2, the enlarged ends have to travel a greater radial distance away from each other compared to FIG. 8B where the enlarged ends 42 and 52 have to travel in a lesser radial distance away from each other when the wire 40 is pulled in an opposing direction from the wire 52. This lesser radial displacement facilitates a smoother relative movement between the enlarged ends 42 and 52 as they disengage.

Thus, the delivery system of the present invention overcomes the above challenges in the following manner. First, it provides quick, reliable and effective mechanical detachment of the coil 100. Second, the self-contained detachment handle uses a perpendicular force to the axial line of the delivery system (hypotube 60) to provide greater stabilization of coil 100 inside the target vessel location. Also, because the detachment handle is self-contained, it does not require an operator to manually connect the elements, so that there is zero risk of connection error. Third, the connection between the coil 100 and the delivery system is very flexible, giving the coil 100 free rotation to allow the coil 100 to flip whenever it gets stuck against the aneurysmal wall and repositions itself to avoid rupture.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A delivery system for an embolic coil, comprising:
a handle housing having a pivotable element that has a front end;
a hypotube having a distal end and a proximal end that is connected to the handle housing, the hypotube having a lumen extending from the proximal end to the distal end, wherein the lumen has a first diameter and a widened section that has a second diameter that is greater than the first diameter;
a first wire having a proximal end and a distal end, with a first enlarged end provided at the proximal end of the first wire and an embolic coil provided at the distal end of the first wire;
a second wire having a proximal end and a distal end, with a second enlarged end provided at the distal end of the second wire and with the proximal end of the second wire connected to the pivotable element;
wherein the first wire and its first enlarged end extend inside the lumen of the hypotube, and the second wire and its enlarged end extend inside the lumen of the hypotube, with the first and second enlarged ends engaging each other; and
wherein the pivotable element is manipulated to pull the second wire in a proximal direction to cause the first and second enlarged ends to disengage when the first and second enlarged ends are both in the widened section.

2. The system of claim 1, wherein the lumen further includes a distal narrowed section and a proximal narrowed section, with the widened section positioned between the distal narrowed section and the proximal narrowed section, and wherein the first and second enlarged ends engage each other at the distal narrowed section during delivery of the embolic coil.

3. The system of claim 2, wherein the diameter of the lumen at the distal narrowed section gradually decreases from the second diameter adjacent the widened section to the first diameter at the distal end of the hypotube, and the diameter of the lumen at the proximal narrowed section gradually decreases from the second diameter adjacent the widened section to the first diameter adjacent the proximal end of the hypotube.

4. The system of claim 1, wherein each of the first and second enlarged ends has a width W, and wherein the diameter of the lumen at the widened section is greater than 2W, but the diameter of the lumen at locations other than the widened section is less than 2W.

5. The system of claim 1, wherein each of the first and second enlarged ends is an olive-shaped end.

6. The system of claim 5, wherein each olive-shaped end has a longitudinal dimension and a width, and the longitudinal dimension is greater than the width, and wherein each olive-shaped end has a completely smooth surface.

7. The system of claim 1, wherein the pivotable element is a lever which is pivoted to pull the second wire in a proximal direction.

8. A delivery system for an embolic coil, comprising:
a handle housing having a pivotable element that has a front end;
a hypotube having a distal end and a proximal end that is adjacent the handle housing, the hypotube having a lumen extending from the proximal end to the distal end;
a first wire having a proximal end and a distal end, with a first olive-shaped end provided at the proximal end of the first wire and an embolic coil provided at the distal end of the first wire;
a second wire having a proximal end and a distal end, with a second olive-shaped end provided at the distal end of the second wire and with the proximal end of the second wire connected to the pivotable element;
wherein the first wire and its first olive-shaped end extend inside the lumen of the hypotube, and the second wire and its second olive-shaped end extend inside the lumen of the hypotube, with the first and second olive-shaped ends engaging each other; and
wherein the pivotable element is manipulated to pull the second wire in a proximal direction to cause the first and second olive-shaped ends to disengage.

9. The system of claim 8, wherein each olive-shaped end has a longitudinal dimension and a width, and the longitudinal dimension is greater than the width, and wherein each olive-shaped end has a completely smooth surface.
